(19) United States
(12) Patent Application Publication
(10) Pub. No.: US 2023/0260618 A1
(43) Pub. Date: Aug. 17, 2023

(54) Body Mechanic app

(71) Applicant: Jonathan David Keller, Mt. Pleasant, SC (US)

(72) Inventor: Jonathan David Keller, Mt. Pleasant, SC (US)

(21) Appl. No.: 17/674,131

(22) Filed: Feb. 17, 2022

(51) Int. Cl. G16H 20/30; A61B 5/00

(52) U.S. Cl. CPC G16H 20/30; A61B 5/45

(57) ABSTRACT

A method and system providing assessment and treatment of musculoskeletal impairments for computers and smart phones is disclosed. A plurality of images and videos for grouping clinical patterns are utilized for which a user matches their symptoms. Once the user determines which grouped symptoms matches their own, they push a button and are directed to identification of a musculoskeletal condition. The user is then lead to treatment in the form of home exercises, rehabilitation tools, proper body mechanic training and postural adjustments. The assessments and treatments are cited with peer reviewed journals.

### BACKGROUND OF THE INVENTION

### Field of Invention

The present disclosure relates generally to apps and websites related to musculoskeletal conditions and impairments. It specifically relates to assessing and treating musculoskeletal injuries with an algorithm based on a system and process of grouping peer reviewed journals and empirical evidence to symptoms of the user for a tailored label of their condition and treatment for educational purposes.

### Background Art Discussion

Computer programs, health websites, and smart phone apps can assist the public in assessment and treatment of musculoskeletal injuries. These entities should ideally provide an accurate injury assessment based on a user's symptoms and an accurate treatment protocol of that impairment. A multiplicity of information is available online for musculoskeletal assessments and treatments. However, there are myriad problems with this information. The information is profoundly scattered, lacking in corroborating research, is not thorough or complete, and is not tailored to the user's symptoms. There are many people that use the internet to search for health care conditions. “More Americans research health information online on an average day than visit health professionals. About 6 million Americans go online for medical advice on a typical day, whereas the American Medical Association estimates that there are an average of 2.75 million ambulatory care visits to hospital outpatient and emergency departments per day and an average of 2.27 million physician office visits per day.”¹ This shows a discrepancy of how many people search for a medical condition online and how many people seek assessment and treatment in person to a medical provider. Navigating this health care information online in a safe and effective manner can be confusing and overwhelming. There is a paucity of research for accurate identification of an injury and a correlating efficacious treatment.² Searching online about a musculoskeletal impairment may be hazardous to your health. Many people try and find an injury diagnosis and treatment online according to a study at Waterloo University in Canada. “When search engines mix correct with incorrect information, we have shown there is potential for significant harm. And when people are desperate, they might see what they want to believe.”³ A study published by the British Journal of Medicine in 2015 titled Evaluation of Symptom Checkers for Self -Diagnosis and Triage, took an in-depth look at symptom checker accuracy and use by testing 23 commonly used symptom checkers. Researchers found that of the 23 symptom checkers analyzed, 34 percent provided the correct initial diagnosis in standardized patient evaluations, 54 percent provided the correct diagnosis out of the first 20 potential diagnoses given, and 57 percent provided the appropriate triage advice (triage advice included recommendations on when to seek emergent care, non-emergent care or self-care).”⁴ Seeking health care for musculoskeletal conditions can be expensive for individuals, employers, and health care insurance companies. According to smallbiztrends.com, (2017) providing health care for their employees was the number one concern among employers.⁵ For the fourth consecutive year, chief executive officers of U.S. companies have reported healthcare costs as their number one economic concern.⁶ After adjustments for price inflation, annual health care spending on inpatient, ambulatory, retail pharmaceutical, nursing facility, emergency department, and dental care increased by $933.5 billion between 1996 and 2013, from $1.2 trillion to $2.1 trillion.⁷Forty percent of Americans had a high deductible in the year 2016 (Under age 65) (1300$, 2600$ considered high deductible amount for health care insurance).⁸ Musculoskeletal impairments cost $213 billion in annual treatment, care and lost wages, according to a new report issued by the United States Bone and Joint Initiative (USBJI).⁹ National health expenditure grew 4.3% to $3.3 trillion in 2016, or $10,348 per person, and accounted for 17.9% of Gross Domestic Product (GDP).¹⁰ There are some Americans that are not able to see a physician or physical therapist due to not having health care insurance. Sometimes they choose not to even seek medical care due to the exceedingly high costs of health care. “The uninsured rate among those aged 19 to 64 increased from 12.7% in 2016 to 15.5% in 2018.”¹¹ “In 2018, 44 percent of Americans said they didn't go to the doctor when they were sick or injured because of financial concerns. Meanwhile, 40 percent said they skipped a recommended medical test or treatment.”¹² Many people in America have musculoskeletal impairments. Eighty percent of Americans will experience an episode of low back pain at some time in their lives.¹³ “Musculoskeletal Disorders (MSDs) affect 1 in 2 adults; In 2012, 126.6 million Americans reported a musculoskeletal injury or condition.”⁹ At the NIH from 1992 until 2018 for alternative forms of health care (clearly not proven for efficacy) the budget has increased from just $2 million to the very hefty $142 million this year.¹⁴ The American public often in obtaining assessment and treatment may not even be receiving accurate assessments and treatments in the traditional health care system in the United States. Up to 50% of physician general practice treatments are not proven with research!!¹⁵ An app that helps a user identify their musculoskeletal condition and corresponding treatment in a user friendly, well organized, thorough, affordable, timely manner based on peer reviewed journals is greatly needed. The present embodiment accomplishes these objectives.

### SUMMARY OF THE DISCLOSURE

The present invention overcomes the previously mentioned problems by providing a user-friendly program that is affordable, well organized, thorough, based on research, and easily accessible via a computer or a smart phone. In accordance with the embodiments of the present invention, its nature is to utilize groupings symptoms that the user matches their own symptoms with. The purpose is for the user to find the symptom cluster that matches their own, click a button, and be directed to treatments of that corresponding musculoskeletal impairment. Advantageously, the program does this at a significantly lower cost than the present systems in place and is exponentially faster to access. The operation is a computer or smart phone system and method to categorize grouped symptoms of the user to a specific and tailored musculoskeletal condition or injury and be directed to corresponding treatments in the form of home exercises, rehabilitation tools, postural education, and body mechanics education.

### BRIEF DESCRIPTION OF THE ELEMENTS

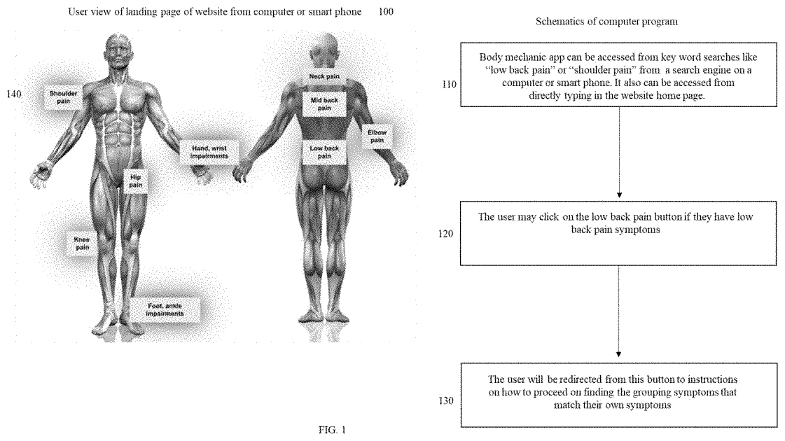

FIG. 1 This is the landing page for the website on the computer and smart phone which shows the various sites of dysfunction to be clicked on

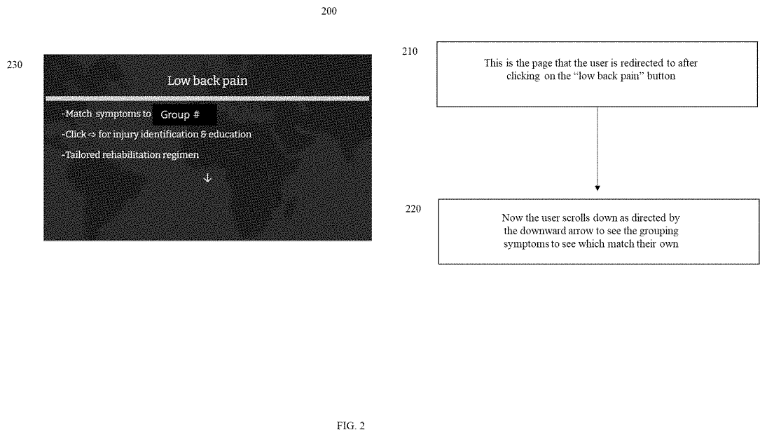

FIG. 2 After an area of pain is clicked upon this is the wording for directions of how to proceed

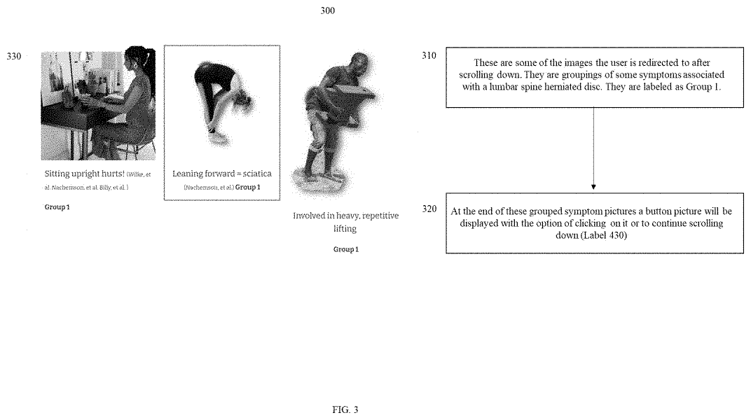

FIG. 3 These are groupings of symptoms in which the user determines if their symptoms match a specific grouping

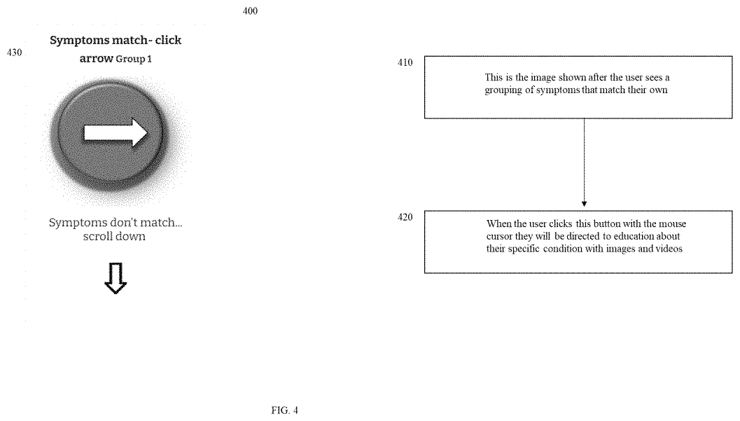

FIG. 4 This is the button that is clicked if the user has symptoms that match a specific grouping

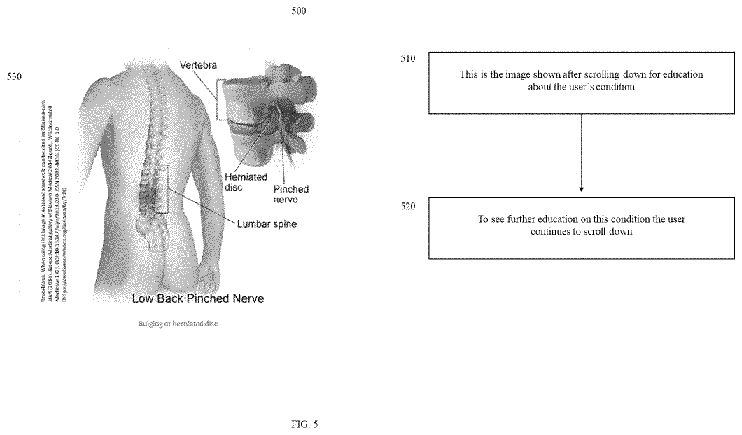

FIG. 5 Identification of the injury or condition

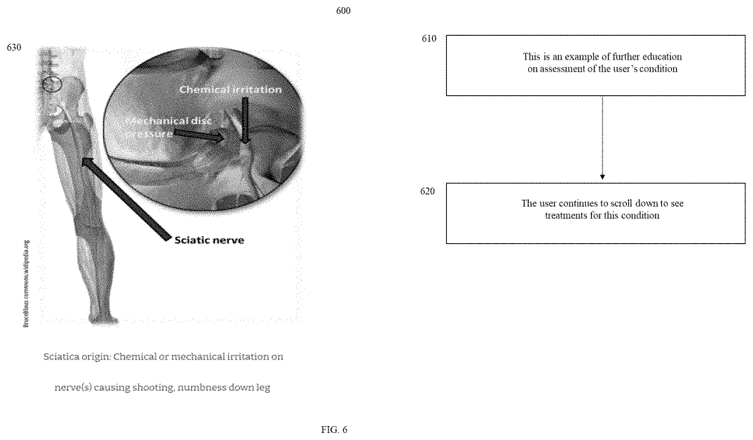

FIG. 6 Further identification of injury or condition

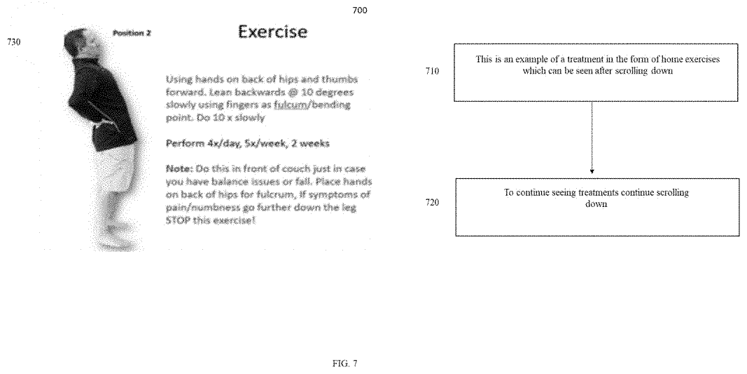

FIG. 7 Treatment in the form of home exercises

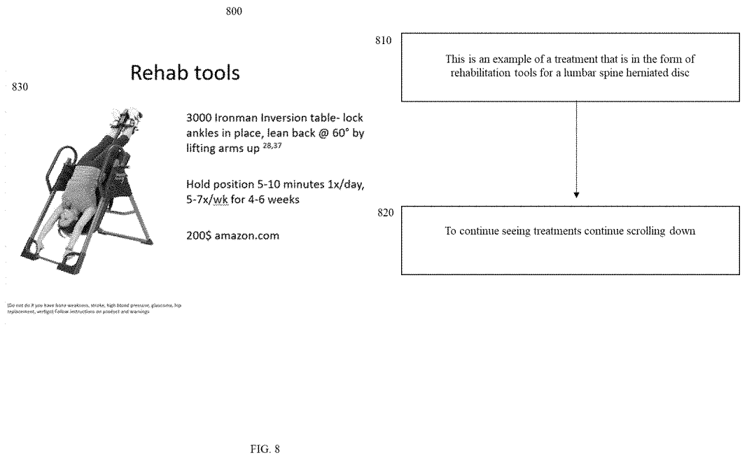

FIG. 8 Treatment in the form of rehabilitation tools use

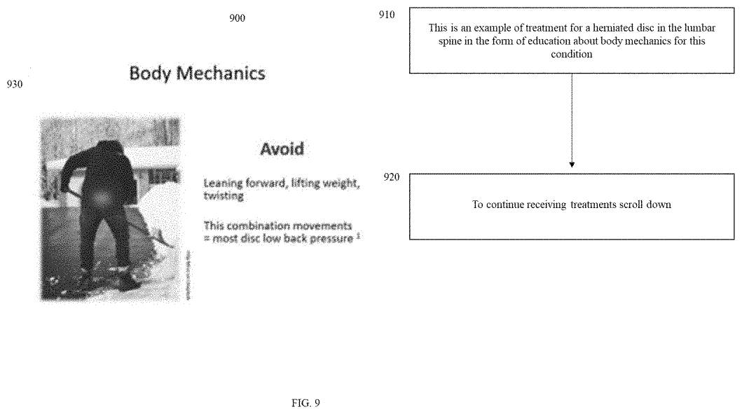

FIG. 9 Treatment in the form of body mechanics education

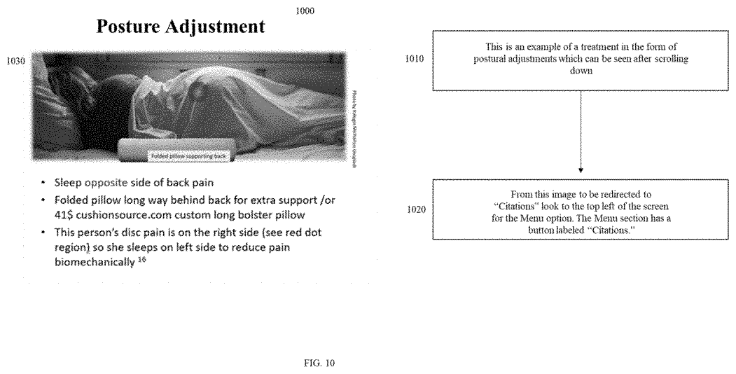

FIG. 10 Treatment in the form of postural adjustments

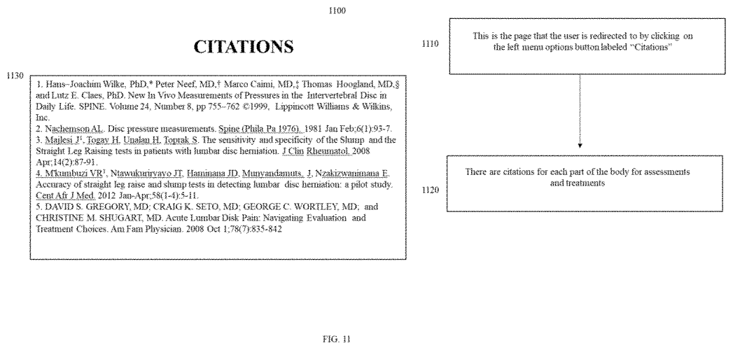

FIG. 11 Citations example in which assessment and treatment are corroborated with peer reviewed journals

### DETAILED DESCRIPTION OF THE ELEMENTS

FIG. 1 presents a block diagram for the construction 100 of an exemplary computer program or smart phone application system in accordance with embodiments of the present invention. The buttons that can be clicked on but are not limited to low back pain, neck pain, shoulder pain, ankle/foot pain, midback, hip pain, knee pain, elbow pain, and hand/wrist pain. Label 110 shows that a user can utilize a search engine tool for combination terms like “low back pain” and “shoulder pain” or type into a toolbar section of a computer website or smart phone search toolbar the Body Mechanic app website address. The label 110 explains that the landing page (label 140) can be accessed via typing in the website address or performing a keyword search on an app store for the smart phone or tablet. Advantageously, by clicking on a button such as “low back pain” the user will be directed to grouped symptoms in the form of pictures to meet the nature of the user's symptoms. This is shown by label 120. At the internet interface 120, advantageously it is connected to label 130 which will direct the user to education about their symptoms to identify their condition. FIG. 140 shows the landing page or first page that occurs after the user inputs keyword terms or combination injury labels like “knee pain” or if the website is directly input into the computer or smart phone.

FIG. 2 presents the architecture (label 200) of a block diagram and front-end image (label 230) explaining what occurs after the user clicks on the button termed “low back pain.” Label 230 has the depiction of what is seen from the front-end user's perspective. This picture shows the user what the directions are for using the program to match their own symptom groupings to the pictures depicted. The block diagram labeled 210 explains that the user is redirected to the label image 230 after clicking on the button “low back pain” from label 140. Label 220 explains for the user to scroll down as directed by the downward arrow on label 230 to see the grouping symptoms to see which match their own.

FIG. 3 presents the architecture (label 300) of an example of a sample of grouped symptoms (Group 1 of the low back section) for a lumbar spine herniated disc. Label 330 is a portion of what is seen by the user after scrolling down on the program. It is a grouping of symptoms that correlate with symptoms consistent with a lumbar spine herniated disc. It is grouping number one for low back pain. Label 310 explains that this is the page that the user is redirected to after scrolling down on low back pain and looks at a variety of groupings to match to their own. Label 320 explains that this is the page that the user is redirected to after clicking on the “low back pain” button.

FIG. 4 is the architecture (label 400) of instructions of how to proceed when the user has symptoms that match their own and to push the click button to identity their musculoskeletal condition, education about this condition, and tailored treatments. It shows a block diagram that shows the front-end user needs to push this button to find out what their diagnosis is and to receive education about it. The label 430 depicts the button to push if their symptoms match the groupings of group 1 which in this case would be a lumbar spine herniated disc. Label 410 explains this is the image shown after the user sees a grouping of symptoms that match their own. Label 420 explains the user clicks this button with the mouse cursor they will be directed to education about their specific condition with images and videos.

FIG. 5 is the architecture (label 500) of directing the user to a picture of the anatomy of their condition and block diagrams of how to proceed to learn more about their condition. It shows a block diagram that shows the front-end of what will occur on the program after scrolling down from the label of 430. The front-end user will see label 530. Advantageously, this label displays the anatomy of a herniated disc of the lumbar spine. Label 510 explains this is the image shown after scrolling down for education about the user's condition. Label 520 explains that in order to obtain more information on this condition the user continues to scroll down.

FIG. 6 is the architecture (label 600) of the image and wording of the condition sciatica which is closely associated often with but not limited to a lumbar spine herniated disc (label 530) and also a block diagram of how the user is to proceed from here. The front-end user will see label 630. Advantageously, this label displays in picture form and easy to understand the anatomy of sciatica and what occurs when the user has sciatica symptoms. This condition is a combination of a lumbar spine herniated disc causing chemical irritation and or mechanical pressure on a nerve root which causes sciatica which is shown on label 630. Label 610 explains that the image seen is further education on the user's condition. Label 620 explains to the user how to navigate to treatments for this condition.

FIG. 7 is the architecture (label 700) of the image and wording for treatment in the form for a lumbar spine herniated disc and or sciatica in the form of a home exercise (label 730) option in standing. Label 710 explains that the image is an example of a treatment in the form of home exercise portion of a program. It directs the user how to correctly perform this activity and how often. Label 720 instructs the user how to proceed for more treatments to help their condition.

FIG. 8 is architecture (label 800) of the image and wording for treatment in the form for a lumbar spine herniated disc and or sciatica in the form of a rehabilitation tool (label 830). Label 810 explains that the image is an example of a treatment in the form of treatment in the form of rehabilitation tool (label 830) to treat a lumbar spine herniated disc. It directs the user how to correctly perform this activity and how often. Label 820 instructs the user how to proceed for more treatments to help their condition.

FIG. 9 is architecture (label 900) of the image and wording for treatment in the form of body mechanics education by way of a block diagram and an image with succinct and easy to understand terminology. The label 930 displays an image displaying treatment in the form of body mechanics. The label 910 explains that this section shows correct body mechanics to help a lumbar spine herniated disc. Label 920 shows what the user to do to see more treatments for their condition.

FIG. 10 is the architecture (1000) in the form of an image and block diagram for treatment of a lumbar spine herniated disc in the form of postural education. The label 1030 shows an image with wording for treatment in the form of postural education for a sleeping position for a lumbar spine herniated disc. Label 1010 explains this is a treatment in the way of postural adjustments. Label 1020 shows the user how to see citations for corroboration on assessment and treatments for musculoskeletal conditions.

FIG. 11 is the architecture (1100) in the form of an image and block diagram for citations for the body mechanic app from peer reviewed journals for assessment and treatment of common musculoskeletal conditions. The label 1130 shows an image with wording these citations. Label 1110 explains that these citations are shown after being directed here after clicking on the menu section for citations. Label 1120 shows that the citations are for corroboration of assessment and treatment of conditions for common injuries.

### CITATIONS

1. Fox S, Lee R, “Vital Decisions: A Pew Internet health report. Main Report: The search for online medical help” May 22, 2002 Pewinternet.org Pew research center internet and technology

2. Krebs P, Duncan D “Health App Use Among US Mobile Phone Owners: A National Survey” JMIR Mhealth Uhealth 2015;3(4):e101

3. Ghenai A, Phd Candidate at Waterloo's David R. Cheriton School of Computer Science and the lead author of the study University of Waterloo, Canada. “Online medical treatment could have dire consequences” April 2018

4. Semigran H, Linder J, Gidengil C, Mehrotra A, Nhcps.com, Evaluation of symptom checkers for self diagnosis and triage: study BMJ 2015; 351 (Published 8 Jul. 2015)

5. Mokdad A H, Forouzanfar M H, Daoud F, et. al, Global burden of diseases, injuries, and risk factors for young people's health during 1990-2013: A systematic analysis for the Global Burden of Disease Study 2013. Lancet. 2016 Jun. 11;387(10036):2383-401.

6. McGinnis M, Olsen L, Bothner K, O'Neill D, and Aisner D, Appendix A “Learning What Works Best: The Nation's Need for Evidence on Comparative Effectiveness in Health Care”: An Issue Overview IOM Roundtable on Evidence-Based Medicine. September 2007 version

7. Dieleman J L, Squires E, Bui A L, “Factors Associated With Increases in US Health Care Spending,” 1996-2013. JAMA. 2017 Nov. 7;318(17):1668-1678

8. Robin A. Cohen, Ph.D., and Emily P. Zammitti, M.P.H., Division of Health Interview Statistics, National Center for Health Statistics. “High-deductible Health Plan Enrollment Among Adults Aged 18-64 With Employment-based Insurance Coverage.” NCHS Data Brief No. 317, August 2018 cdc.gov

9. Katz S, Weinstein S, Yelin E, Steering Committee “The Burden of Musculoskeletal Diseases in the United States Prevealence, Societal and Economic Cost,” Burden of Musculoskeletal Diseases in the United States (BMUS) United States Bone and Joint Initiative 2014-2015 3^{rd} edition

10. Cms.gov The National Health Expenditure Accounts (NHEA) Centers for Medicare and Medicaid Services Research Statistics

11. Collins S, Gunj a M, Doty M, Bhupal H, “First Look at Health Insurance Coverage in 2018 Finds ACA Gains Beginning to Reverse” To the Point Quick Takes on Health care policy and practice. Mar. 30, 2018 Commonwealthfund.org

12. Ingersoll T, “New Survey Finds Large Number of People Skipping Necessary Medical Care Because of Cost” Mar. 26, 2018 Westhealth.org NORC at the University of Chicago

13. Rubin D I, “Epidemiology and risk factors for spine pain.” Neurol Clin. 2007;25(2):353-371.

14. Salzberg, “$142 Million For Quack Medicine Buried Inside The New 2018 Budget”, Forbes.com 2018

15. Garrow J, HealthWatch “How much of orthodox medicine is evidence based?” BMJ. 2007 Nov. 10; 335(7627): 951

What is claimed:

1. A novel method for assessment and treatment of musculoskeletal impairments comprising: a computer-generated software program that incorporates a proprietary algorithm which interfaces with an application for smart phones and computers; means of categorizing symptoms of a user that may be consistent with human body impairments and correlating treatments for an individual by applying specialized medical knowledge, utilizing the steps of accessing a computer program by means of a computer or smartphone which is not in electronic communication with an exercise or therapy device associated with the individual; selecting one body region out of all the general displayed body part regions divided into areas but not limited to the low back, knee, hip, neck, midback, elbow, wrist/hand, shoulder, foot/ankle, and shin to be assessed and treated from a computer-generated menu of options; means of generating symptom categories based on which body part is selected and the user matching their own symptoms to several groupings presented by the program; and a means for generating a representation of identification of that musculoskeletal impairment; means of generating tailored treatments in the form of home exercises, rehabilitation tools, body mechanics, and postural corrections for the user based on their particular grouping of symptoms they select.

2. The method according to claim 1, wherein the program is set in motion after the user clicks on the area of the body button of their interest or injury for education about that region; generation of a plurality of representations that are assembled together in several grouped symptoms at which point the user clicks on another button if the user's symptoms match a particular group.

3. The method according to claim 2, wherein the user receives the identification of their musculoskeletal impairment(s) and the anatomy of that condition which is extremely useful for the user.

4. The method according to claim 1, wherein a user receives non-obvious and currently not easily accessible and valid source information in the form of musculoskeletal condition representation of efficacious treatment(s) that correlates to their condition in the form of home exercises that are structured with but not limited to directions for accuracy of form, safety in performance, frequency of use, and time frames of proper use.

5. The method according to claim 1, wherein a user is directed to education about their musculoskeletal condition representation of effective treatment(s) that correlates to that condition structured but not limited to recommended rehabilitation tools with proper use, proper duration, and where and how to access this rehabilitation device.

6. The method according to claim 1, wherein a user is directed to their musculoskeletal condition representation of treatment that correlates to that condition structured but not limited to proper body mechanics.

7. The method according to claim 1, wherein a user is directed to education about their musculoskeletal condition structured but not limited to the representation of effective treatment(s) that correlates to that condition in the form of postural re-education with proper musculature activation and proper tools to aid the user immensely.
